(19) United States
(12) Patent Application Publication
(10) Pub. No.: US 2008/0058600 A1
(43) Pub. Date: Mar. 6, 2008

(54) Method and system for stabilizing an optical imaging fiber in a diagnostic scope

(76) Inventor: David Bowman, Marietta, GA (US)

Correspondence Address: John Doughty, 42 Creekside Court, Acworth, GA, 30101, US

(21) Appl. No.: 11/897,453

(22) Filed: Aug. 30, 2007

Related U.S. Application Data
(60) Provisional application No. 60/841,315, filed on Aug. 31, 2006.

(51) Int. Cl. A61B 1/07

(52) U.S. Cl. 600/146

(57) ABSTRACT

An adhesive is applied to a composite fiber bundle to bond the bundle with respect to the housing of a docking assembly. The docking assembly is removably secured to a catheter steering assembly. The composite fiber bundle is also fixed at the scope end of the fiber cable, which includes the composite bundle, so movement of the distal end of the image fiber with respect to a lens at the scope end of the fiber cable is resisted unless medical personnel operating the steering assembly intentionally move the steering assembly.

### CROSS REFERENCE TO RELATED APPLICATION

This application claims priority under 35 U.S.C. 119(e) to U.S. provisional patent application No. 60/841,315 entitled “Method and system for stabilizing an optical imaging fiber in a diagnostic scope,” which was filed Aug. 31, 2006, and is incorporated herein by reference in its entirety

### BACKGROUND

A fiber optic imaging system typically consists of a light source, a digital camera with a remote CCD housing, a monitor and an image capture device. The image capture device contains a distal lens system and both an image bundle and light fibers for illuminating and transmitting an image of anatomy being investigated back to the remote CCD. The image bundle in turn consists of a plurality of glass or plastic fibers wherein each individual fiber creates an image pixel. These fibers are typically bound together and clad in a sheath. Both the image and light fibers are usually clad in an additional sheath to hold and protect the delicate combined assembly.

As discussed above, the light fibers transmit light from a light source via a light cable and into the fiber optic assembly for delivery to the target anatomical location. The target location reflects light to form an image that is captured through a lens system by the image fiber. The reflected image is transmitted to the camera via the image fibers, captured and digitized so that the image of the target location may be displayed on the monitor. This process is known in the art, in particular the medical art fields as evidenced by the prevalent use of fiber optic based endoscopes and arthroscopes.

Some fiber optic endoscope or arthroscopic devices are steered by use of a steering catheter or other hand held device. This allows the surgeon greater flexibility in navigating to and targeting the objective anatomy. Such a device may also be used to deliver infusions, such as isotonic saline, medications, a mechanical instrument or even an energy delivering device such as RF or laser.

The outer protective sheath of the image and light fiber optic bundle must be able to protect the delicate assembly that includes the light and image fiber bundles during normal surgical use and subsequent cleaning and sterilization processes. Materials such as polyimide coated wire braiding are typically used for the protective sheath. Because of the typical length of the catheter and the small diameters of the fibers, the optical fiber strands within the sheathing material is often loose. This ensure that the fibers are not damaged during manufacture and allows for bending during normal use by the surgeon. In particular, individual glass fibers, if restricted by being fixed to one another for the entire interior length of the sheathing, would likely break with just the smallest bending motion, thus negating the benefits of a flexible endoscope or arthroscope. Typical manufacture consists of fixing the lens system, which is at a distal end of the outer sheath with respect to a remote steering/navigation assembly, to the image and light fibers. The proximal end of the fiber sheath is fixed to the housing of the scope interface lens system steering/navigation assembly.

However, a problem often encountered with typical steering/navigation system devices as known in the art is that while the fiber optic bundle is flexed and/or moved around during performance of a medical procedure, it may rotate with respect to the outer sheath and the scope housing. Since the image capture device is typically fixed with respect to the protective sheathing, the image that the surgeon performing the procedure sees may rotate as the fiber rotates with respect to the sheath.

The loss of image orientation during the course of a surgical procedure often causes the surgeon to become confused as to the orientation of the image he or she is looking at. This may result in lost time while the surgeon regains orientation, or, in a worst case scenario, trigger an energy device at the incorrect anatomical structure. Such a scenario could seriously jeopardize the safety of the patient. Thus, there is a need in the art for method and system for preventing the orientation of the image the surgeon sees from changing during the course of a given surgical procedure.

### SUMMARY

A remote docking assembly is fixed along the fiber optic cable protective sheath. The docking assembly can be locked onto the steering catheter that is used to navigate the fiber optic endoscope or arthroscope. Into and within the docking mechanism, a bonding material is introduced, or applied, to hold and fix the image fiber to the remote docking assembly, thus preventing the image fiber from rotating free of the outer sheath material. Such a docking mechanism may include the use of locking pins, for example, that engage with a J-shaped hook-slot, or similar means, of a steerable catheter to removably secure the docking assembly to the steering assembly housing. Empirical testing has shown this bonding method to maintain image orientation despite extensive manipulation of an endoscope or arthroscope coupled to a docking station to which the bonding method is applied.

### DECRIPTION OF THE DRAWINGS

FIG. 1 illustrates a steering/navigation assembly with a connector for connecting a remote docking assembly.

FIG. 2 illustrates a cutaway view of image lens system that focuses an image on the ends of a optical fiber bundle at the distal end of fiber cable.

FIG. 3 illustrates a scope housing at a proximal end of a optical fiber cable with a docking assembly at a mid portion of the cable.

FIG. 4 illustrates a cutaway of a remote docking assembly.

FIG. 4a illustrates an adhesive and an adhesive surface of a composite fiber bundle.

### DETAILED DESCRIPTION

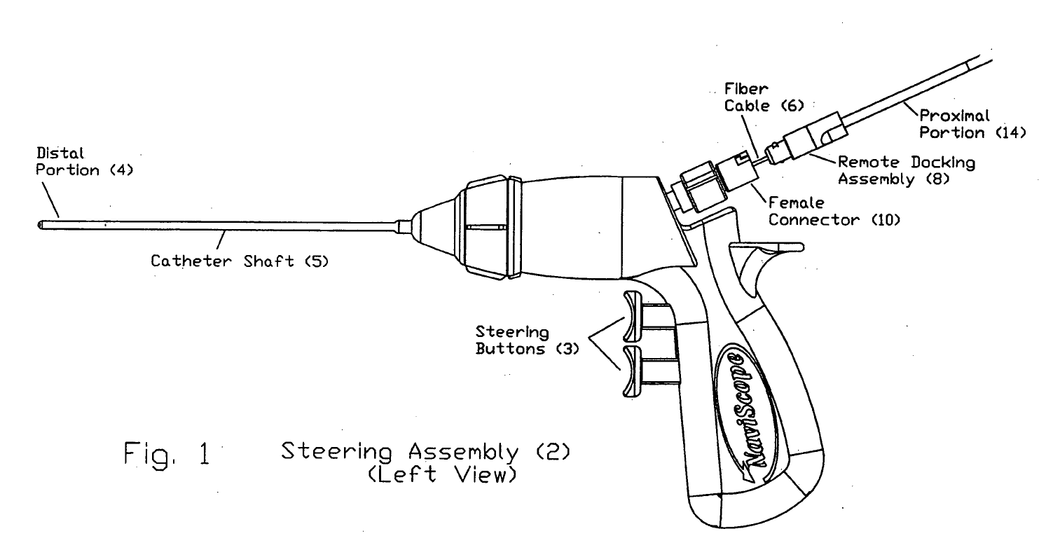

Turning now to the figures, FIG. 1 illustrates a steering assembly 2 that is contoured to fit in a hand. Steering assembly 2 has steering buttons 3 for controlling the movement of a lens system (attached to fiber cable) at the distal portion 4 of catheter shaft 5. Fiber cable 6 is shown in the figure between the remote docking assembly 8 and female connector 10. Remote docking assembly 8 may removably secure fiber cable 6 and proximal portion 14 to steering assembly 2. Remote docketing assembly 8 and female connector 10 may be made of various materials, including metal and/or plastic, and may operate similarly to a BNC connector having a J-shaped hook-slot known in the electrical arts to connect a cable to a device, for example.

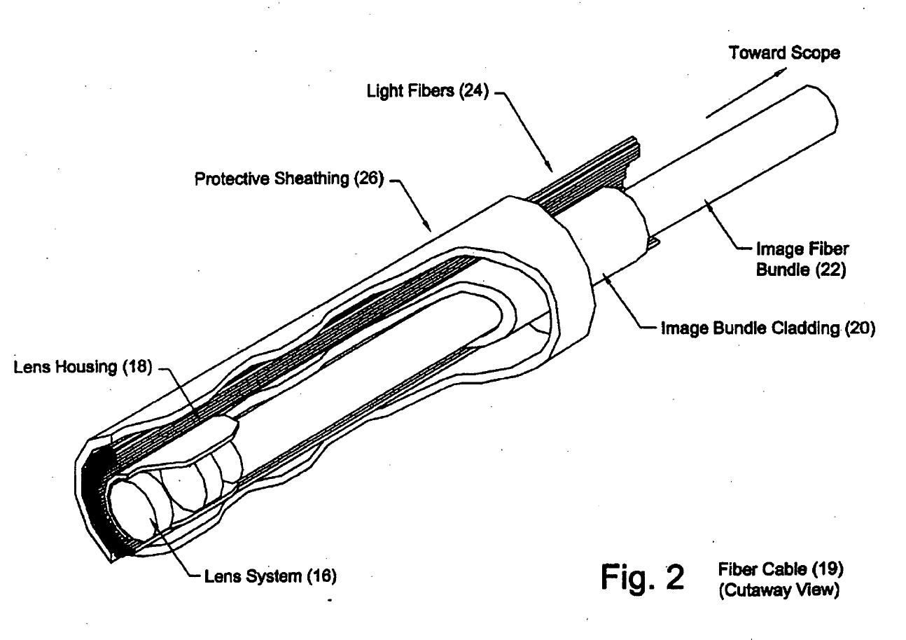

Turning now to FIG. 2, a lens system 16 is housed inside lens housing 18 at a distal end 19 of the fiber cable 6. Lens housing 18 is coupled to image bundle cladding 20, which separates and shields image fiber bundle 22 from light fibers 24. Light fibers 24 typically carry light from a light source, typically from a scope assembly as described elsewhere herein. Protective sheathing 26 surrounds light fibers 24 to contain them and protect them from the environment being investigated during a medical procedure, or other use of which a scope assembly having the components just described is made.

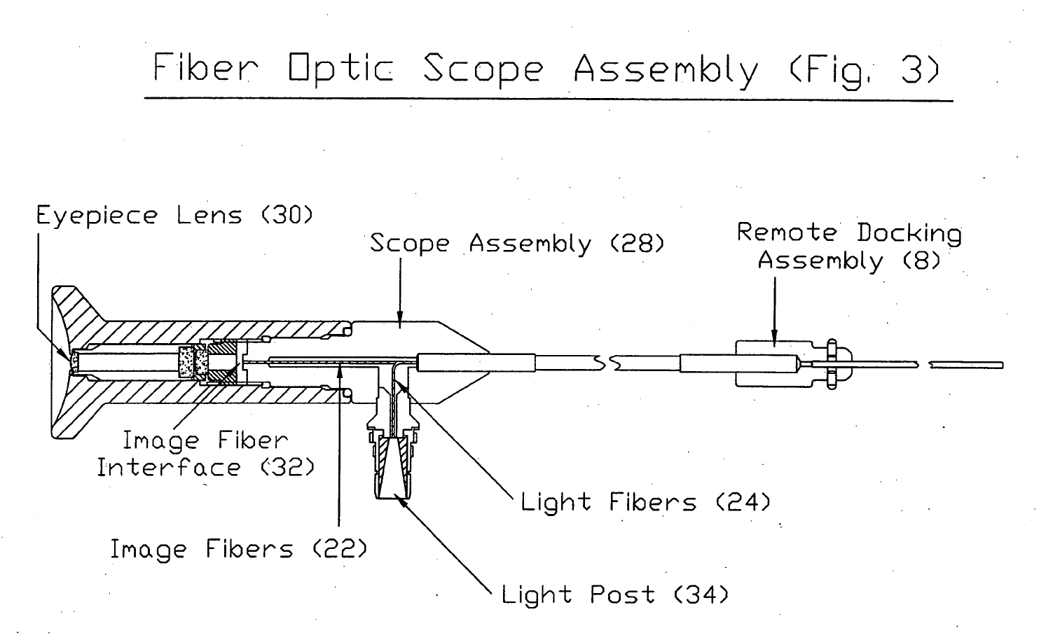

Turning now to FIG. 3, a scope assembly 28 is shown. A surgeon or other medical personnel views an image of the location being investigated by looking into eyepiece lens 30, through which the image is focused from fiber interface 32. Image fiber interface 32 is optically, as well as physically, coupled to image fibers 22. Light is introduced into light carrying fibers 24 via light post 34, which is adapted to receive a light source coupled thereto. Light fibers 24, image fibers 22, and the cladding and sheathing as described in reference to FIG. 2, pass through remote docking assembly 8.

Remote docking assembly 8, as discussed above in reference to FIG. 1, is secured to steering assembly 2 via locking pins or other detachable securing means, such as a threaded coupling, snap lock coupling or other detachable coupling. When remote docking station 8 is secured to steering mechanism 2, manipulating steering buttons on the steering assembly moves internal mechanisms coupled thereto to cause catheter shaft 5, which contains image fibers 22 as discussed above in reference to FIG. 3, to move in response thereto. By substantially rigidly linking the image fibers to the housing of remote docking assembly 8, the orientation relationship between the image fibers and the steering assembly is maintained, because the remote docking assembly is mechanically coupled to the steering assembly and the image fibers.

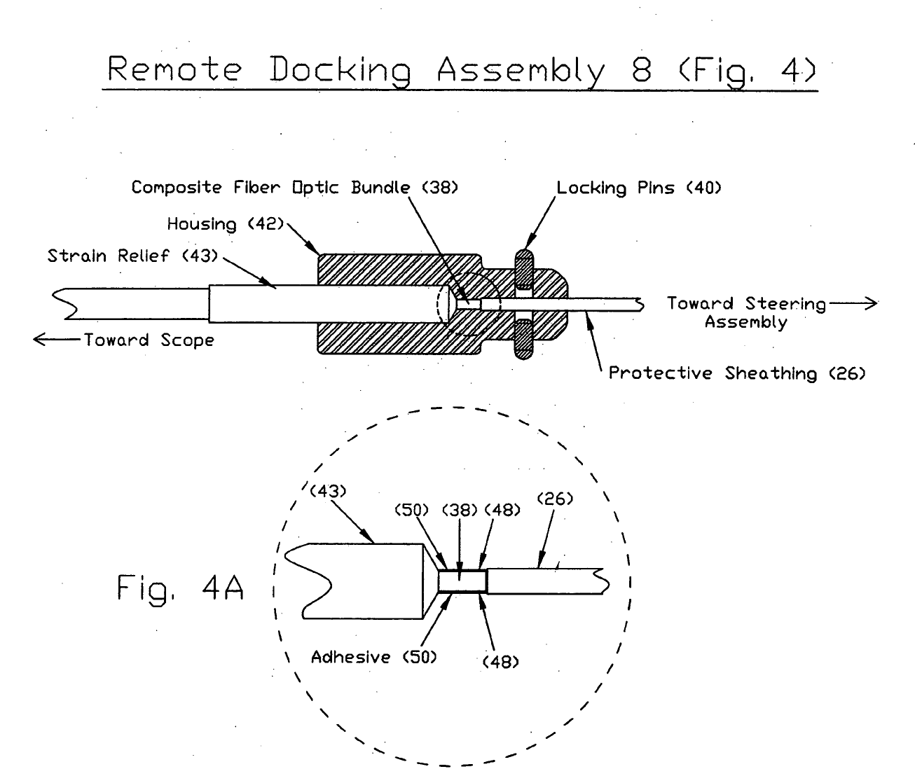

Turning now to FIG. 4, a cutaway view of remote docking assembly 8 is shown. As discussed in reference to FIG. 1, lock pins 40 similar to those used on the male portion of an electrical BNC connector are located in docking assembly housing 42, which may be made out of a variety of rigid, or semi-rigid material, such as, for example metal or plastic. Thus, lock pins 40 serve to removably secure housing 42 to female portion 10 as described in reference to FIG. 1. The entire bundle of fibers, including the image fibers, image fiber cladding, light fibers, as well as the protective sheathing, referred to as items 22, 20, 24 and 26 respectively in FIG. 2, is hereinafter referred to as composite fiber bundle 38.

FIG. 4A shows an expanded portion of the area between strain relief 43 and the lock pins 40. A portion of outer protective sheathing 26, is removed to expose fiber surface 48. As shown in earlier figures, fiber adhesive surface 48 of composite fiber bundle 38 typically comprises light fibers 24 surrounding image bundle cladding 20 that in turn surround image fiber bundle 22 as illustrated in reference to FIG. 2. Continuing with discussion of FIG. 4A, an adhesive is introduced, or applied, onto adhesive surface 48 of composite bundle 38, the adhesive surface referring to the fibers and surfaces thereof of fibers that comprise the light fibers 24. Adhesive 50 fixes composite bundle 38 to housing 42 of remote docking assembly 8. It will be appreciated that adhesive 50 may be introduced via a temporary fixture that preferably annularly surrounds surface 48 so that adhesive may be received from a supply of adhesive, such as a tube or other injection means, at a single injection point of the fixture, under pressure. The temporary fixture distributes adhesive 50 under pressure to surround adhesive surface 48 and image fiber cladding 20 under surface 48 (the term under referring to being generally closer to the axis of bundle 38 with respect to surface 48). Alternatively, adhesive 50 may be manually applied preferably annularly around surface 48. In yet another aspect, adhesive 50 may be applied at a point on surface 48, or partially annularly around adhesive surface 48, but not completely around the surface. As shown in FIG. 4, adhesive 50 may be applied to all of exposed surface 48, as shown between strain relief 43 and sheathing 26 in the figure. Alternatively, adhesive 50 may also be applied only at the transition from the light fibers comprising the adhesive surface 48 and sheathing 26.

After, introducing, or applying, adhesive 50 to surface 48 of composite bundle 38, the adhesive permeates void space surrounding the light fibers, thus securing image cladding 20, as shown in reference to FIG. 2 to housing 42 of docking assembly 8. Therefore, when medical personnel perform a procedure and manipulate catheter 5, as shown in FIG. 1, the fiber cable moves in lockstep with the catheter because the catheter is coupled to the steering assembly and the fiber cable is fixed to the docking assembly housing. Thus, an image viewed through eyepiece lens 30 shown in FIG. 3 does not change orientation with respect to scope assembly 28.

What is claimed is:

1. A system for reducing image rotation in a diagnostic imaging device, comprising:
a docking assembly having a housing fixed to image cladding of a composite fiber bundle that includes an image fiber bundle.

2. The system of claim 1 wherein the composite fiber bundle further comprises a light delivery fiber bundle surrounding the image fiber bundle.

3. The system of claim 2 wherein the means for fixing surrounds the light delivery fiber bundle.

4. The system of claim 1 wherein the means for fixing is an adhesive.

5. The system of claim 4 wherein the adhesive is an epoxy adhesive.

6. The system of claim 3 wherein the means for fixing is annularly introduced around the light delivery fiber bundle at an outer surface thereof.

7. A system for reducing image rotation in a diagnostic imaging device having a docking assembly having a housing fixed to image fiber cladding of a composite fiber bundle that includes an image fiber bundle, the docking assembly made using a method comprising the step of: fixing the composite fiber bundle with respect to the housing such that the image fiber bundle does not rotate with respect the housing.

8. The system of claim 7 wherein the composite fiber bundle further comprises a light delivery fiber bundle surrounding the image fiber bundle.

9. The system of claim 8 wherein the composite fiber bundle is fixed to the docking assembly by an adhesive.

10. The system of claim 9 wherein the adhesive is applied to an annular surface of the composite fiber bundle.

11. The system of claim 9 wherein the adhesive is an epoxy adhesive.

12. The system of claim 9 wherein the annular surface is on the light delivery fiber bundle.

13. The system of claim 9 wherein the adhesive is applied annularly around the entire annular surface.

14. The system of claim 9 wherein the adhesive is applied at a point on the annular surface.
